(19) United States
(12) Patent Application Publication
(10) Pub. No.: US 2010/0184729 A1
(43) Pub. Date: Jul. 22, 2010

(54) New Pharmaceutical Compositions for Treatment of Thrombosis

(75) Inventors: Paul A. Reilly, Danbury, CT (US); James C. Gilbert, Bethlehem, CT (US); Thomas H. Mueller, Danbury, CT (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 12/727,933

(22) Filed: Mar. 19, 2010

Related U.S. Application Data
(63) Continuation of application No. 11/277,503, filed on Mar. 26, 2006.

(30) Foreign Application Priority Data
Mar. 29, 2005 (EP) 05006711

(51) Int. Cl. A61K 31/616; A61K 31/4365; A61P 7/02; A61P 9/10

(52) U.S. Cl. 514/161; 514/301

(57) ABSTRACT

The present invention relates to novel pharmaceutical compositions comprising at least one direct thrombin inhibitor and at least one additional active compound selected from the groups consisting of platelet inhibitors, low molecular weight heparins (LMWH) and heparinoids as well as unfractionated heparin, factor Xₐ inhibitors, combined thrombin/factor Xₐ inhibitors, fibrinogen receptor antagonists (glycolprotein IIb/IIa antagonists) and Vitamin K antagonists, optionally together with one or more pharmaceutically acceptable excipients or carriers for the treatment of thrombosis.

### RELATED APPLICATIONS

This application claims priority to co-pending U.S. application Ser. No. 11/277,503, filed Mar. 26, 2006, which claims priority to European Patent Application No. 05 006 711.5, filed Mar. 29, 2005, the contents of which are incorporated herein by reference in their entirety.

### FIELD OF THE INVENTION

The present invention relates to novel pharmaceutical compositions comprising one or more, preferably one, direct thrombin inhibitors (DTI) 1, and at least one additional active compound 2, processes for preparing them and their use as medicament in the treatment of thrombosis.

### DETAILED DESCRIPTION OF THE INVENTION

In a first aspect, the present invention relates to pharmaceutical compositions comprising at least one direct thrombin inhibitor 1 selected from the group consisting of
(1.1) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (dabigatran) having the following structure

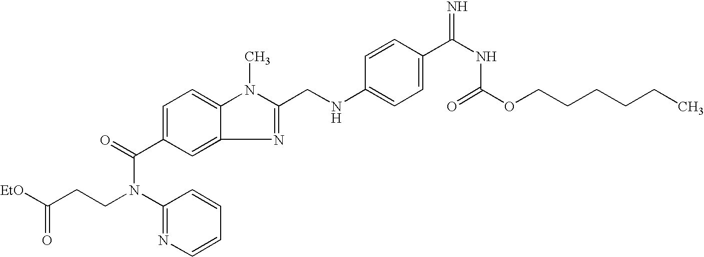

(1.2) 1-methyl-2-(4-amidinophenylaminomethyl)-benzimidazol-5-yl-carboxylic acid-(N-2-pyridyl-N-2-hydroxycarbonylethyl)-amide having the structure

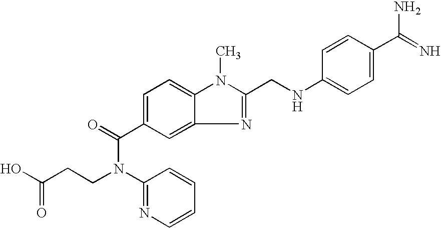

(1.3) 1-methyl-2-[4-(N-hydroxyamidino)-phenylaminomethyl]-benzimidazol-5-yl-carboxylic acid-(N-2-pyridyl-N-2-ethoxycarbonylethyl)-amide having the structure

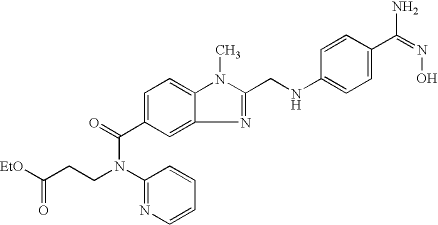

(1.4) melagatran (inogatran),
(1.5) ximelagatran,
(1.6) hirudin,
(1.7) hirolog,
(1.8) argatroban,
   optionally in the form of tautomers, racemates, enantiomers, diastereomers, pharmacologically acceptable acid addition salts, solvates or hydrates, prodrugs thereof,
   and further comprising one or more additional active compounds 2 selected from the groups consisting of platelet inhibitors 2a, low molecular weight heparins (LMWH) and heparinoids as well as unfractionated heparin 2b, factor Xₐ inhibitors 2c, combined thrombin/factor Xₐ inhibitors fibrinogen receptor antagonists (glycoprotein IIb/IIa antagonists) 2e and Vitamin K antagonists 2f, optionally together with one or more pharmaceutically acceptable excipients or carriers. All active components should be present in effective amounts.

The active compounds 1.1 to 1.3 are disclosed in the prior art, e.g. in WO 98/37075 and WO 04/014894.

Prodrugs of the drugs mentioned above are such derivatives containing one or more groups capable of being cleaved in vivo, particularly a group which can be converted in vivo into a carboxy group or/and a group capable of being cleaved in vivo from an imino or amino group. Compounds containing two groups capable of being cleaved in vivo are so-called double prodrugs. Groups which can be converted in vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

In the pharmaceutical compositions according to the present invention, the direct thrombin inhibitors 1 may be contained in a form selected from tautomers, optical isomers, enantiomers, racemates, diastereomers, pharmacologically acceptable acid addition salts, solvates or hydrates, as far as such forms exist, depending on the individual compound. Pharmaceutical compositions comprising one or more, preferably one, compound 1 in form of a substantially pure enantiomer are preferred.

Pharmacological acceptable acid addition salts of direct thrombin inhibitors 1 comprise salts selected from the group consisting of the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydrooxalate, hydrosuccinate, hydrobenzoate and hydro-p-toluolsulphonate, preferably hydrochloride, hydrobromide, hydrosulphate, hydrophosphate, hydromaleate, hydrofumarate and hydromethansulphonate. Some of the compounds 1 may add more than one equivalent acid, e.g. two equivalents. The salts of hydrochloric acid, methanesulphonic acid, maleic acid, benzoic acid and acetic acid are especially preferred. The most preferred salt of 1 is the methansulfonic acid addition salt.

The pharmaceutical compositions according to the invention comprising at least one direct thrombin inhibitor 1 and at least one additional active compound 2 are not restricted to binary combinations of actives. The combinations disclosed exemplary below comprising an direct thrombin inhibitor 1 together with an additional active compound 2 may comprise a third or a third and a fourth, preferably a third active compound, also selected from the group consisting of platelet inhibitors 2a low molecular weight heparins and heparinoids 2b, factor Xₐ inhibitors 2c, combined thrombin/factor Xₐ inhibitors 2d, fibrinogen receptor antagonists (glycoprotein IIb/IIa antagonists) 2e and Vitamin K antagonists 2f. All components 2a to 2f mentioned specifically hereinafter are described in the prior art.

In a first preferred embodiment of the invention the pharmaceutical combination is binary, comprising an direct thrombin inhibitor 1 and an active compound selected from one of the classes 2a, 2b, 2c, 2d, 2e and 2f. A preferred binary combination contains compound 1.1 and either clopidogrel or acetylsalicylic acid (ASA).

In a second preferred embodiment of the invention the pharmaceutical combination is ternary, comprising an direct thrombin inhibitor 1, and two compounds selected from the classes 2a, 2b, 2c, 2d, 2e and 2f, while the additional two compounds may belong to one and the same or two different classes selected from 2a, 2b, 2c, 2d, 2e and 2f. Preferably both additional compounds are selected from class 2a. A preferred ternary combination contains compound 1.1, clopidogrel and acetylsalicylic acid.

In a third embodiment of the invention the pharmaceutical combination is quarternary, comprising two direct thrombin inhibitors 1 and two active compounds selected from either one or from two different classes of 2a, 2b, 2c, 2d, 2e and 2f, preferably selected from either one or from two different classes of 2a, 2b and 2e.

Any reference to a direct thrombin inhibitor 1 within the scope of the present invention should be understood as a reference to any specific direct thrombin inhibitor selected from compounds 1.1 to 1.8. above-mentioned. Analogously, any reference to an active compound selected from the classes 2a, 2b, 2c, 2d, 2e and 2f within the scope of the present invention should be understood as a reference to any active compound of these classes mentioned specifically below.

In the pharmaceutical combinations according to the invention the active substances may be combined in a single preparation, e.g. as a fixed dose combination comprising the active ingredients in one formulation together, or contained in two or more separate formulations, e.g. as a kit of parts adapted for simultaneous, separate or sequential administration. Pharmaceutical compositions containing the active substances 1 and 2 in a single preparation are preferred according to the invention.

In all embodiments of the invention, the direct thrombin inhibitors 1.1 is preferred, especially in form of its acid addition salt with methanesulfonic acid.

All pharmaceutical compositions of the present invention can be advantageously used in the following indications:

for the prevention and treatment of the consequences of thrombotic and thromboembolic diseases such as
deep vein thrombosis (DVT) pulmonary embolism, and other venous thrombotic events in patients at risk for such events (post-orthopedic surgery, medical patients, cancer patients, surgical patients),
stroke prevention in atrial fibrillation (SPAF),
stroke prevention in other populations at high risk for such events (heart failure or left ventricular dysfunction, high risk patients with myocardial infarction, patients with valve disease or valve replacement)
thrombosis and thombotic events in patients with acute myocardial infarction or acute coronary syndromes, including patients undergoing thrombolysis or those with stents or percutaneous coronary intervention (PCI), or both,
post-myocardial infarction (MI), in patients who have received thrombolysis or those with percutaneous coronary intervention or post coronary bypass surgery,
or other acute coronary syndromes
for prevention or treatment of thrombosis, in particular for treatment of patients with stents or percutaneous coronary intervention (PCI).

Preferred fields of application are chronic and acute thromboembolic diseases or events.

Particularly preferred fields of application are DVT and SPAF.

Thus a second aspect of the invention is a method of treating any of the indications mentioned hereinbefore comprising administering to a patient in need thereof a pharmaceutical composition according to the invention, comprising at least one of the selected direct thrombin inhibitors 1 in combination with one or more additional active compounds 2 selected from the groups consisting of platelet inhibitors 2a low molecular weight heparins and heparinoids as well as unfractionated heparin 2b, factor Xₐ inhibitors 2c, combined thrombin/factor Xₐ inhibitors 2d, fibrinogen receptor antagonists glycoprotein IIb/IIa antagonists) 2e and Vitamin K antagonists 2f, optionally together with one or more pharmaceutically acceptable excipients. The expression “patient” is meant to comprise the mammal animal body, preferably the human body. The method of treatment is meant to encompass simultaneous as well as successive administration of the active components.

A third aspect of the invention is the use of any of the selected direct thrombin inhibitors 1 in combination with one or more additional active compounds 2 selected from the groups consisting of platelet inhibitors 2a, low molecular weight heparins and heparinoids as well as unfractionated heparin 2b, factor Xₐ inhibitors 2c, combined thrombin/factor Xₐ inhibitors 2d, fibrinogen receptor antagonists (glycoprotein IIb/IIa antagonists) 2e and Vitamin K antagonists 2f, optionally together with one or more pharmaceutically acceptable excipients, for the manufacture of a pharmaceutical composition for treating any of the indications mentioned hereinbefore in a patient in need thereof. This aspect encompasses the preparation of all pharmaceutical compositions according to the invention mentioned hereinbefore or below.

Preferred embodiments of the pharmaceutical compositions of the invention as well as the indications to be treated apply analogously regarding to the second and third aspect of the invention.

### Pharmaceutical Compositions Comprising a Direct Thrombin Inhibitor 1 and a Platelet Inhibitor 2a:

One embodiment of the invention is a pharmaceutical composition comprising an direct thrombin inhibitor 1 and a platelet inhibitor 2a. Binary compositions containing only one active 1 and one active 2a, optionally together with one or more pharmaceutically acceptable excipients or carriers, are preferred. In the pharmaceutical combinations according to the invention preferred platelet inhibitors 2a are selected from the group consisting of acetylsalicylic acid 2a.1, clopidogrel 2a.2 and ticlopidine 2a.3, optionally in the form of the racemates, the enantiomers, the diastereomers and optionally the pharmacologically acceptable acid addition salts and the hydrates thereof.

According to the instant invention more preferred platelet inhibitors 2a are selected from the group consisting of acetylsalicylic acid 2a.1, clopidogrel 2a.2 and ticlopidine 2a.3, optionally in the form of the racemates, the enantiomers, the diastereomers and optionally the pharmacologically acceptable acid addition salts and the hydrates thereof.

Examples of pharmacologically acceptable acid addition salts of the platelet inhibitors 2a according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid, 4-phenylcinnamic acid, 5-(2,4-difluorophenyl)salicylic acid or maleic acid. If desired, mixtures of the abovementioned acids may also be used to prepare the salts of 2a.

According to the invention, the salts of the platelet inhibitors 2a selected from among the hydrochloride, hydrobromide, sulphate, phosphate, fumarate, methanesulphonate, 4-phenylcinnamate, 5-(2,4-difluorophenyl)salicylate, maleate and xinafoate are preferred.

In the pharmaceutical compositions according to the invention, the compounds 2a may be present in the form of their racemates, enantiomers or mixtures thereof. The separation of the enantiomers from the racemates may be carried out using methods known in the art (e.g. by chromatography on chiral phases, etc.).

Besides therapeutically effective quantities of 1 and 2a the pharmaceutical compositions may contain in addition a pharmaceutically acceptable carrier. The present invention encompasses both pharmaceutical compositions with or without pharmaceutically acceptable carriers.

Especially preferred pharmaceutical compositions according to the invention comprise the following specific combinations of direct thrombin inhibitors 1 and platelet inhibitors 2a, either as free bases or pharmacologically acceptable acid addition salts:

1.1 and 2a.1, 1.1 and 2a.2, 1.1 together with both 2a.1 and 2a.2,
particularly preferred are pharmaceutical compositions comprising
the methanesulfonate of 1.1 and 2a.1,
the methanesulfonate of 1.1 and 2a.2,
the methanesulfonate of 1.1 together with 2a.1 and 2a.2.

The proportions in which the active substances 1 and 2a may be used in the active substance combinations according to the invention are variable. Active substances 1 and 2a may possibly be present in the form of salts, solvates or hydrates. Depending on the choice of the compounds 1 and 2a, the weight ratios which may be used within the scope of the present invention vary on the basis of the different molecular weights of the various salt forms. The pharmaceutical combinations according to the invention may contain 1 and 2a generally in ratios by weight ranging from 10:1 to 1:15, preferably from 8:1 to 12:1, e.g. 1:1 to 1:10 or 2:3.

If not specified otherwise, the weights and the weights ratios specified hereinbefore and below are based on the free bases of the actives.

For example, pharmaceutical compositions according to the invention usually contain a quantity of 1.1 per single dose between about 50 mg and 200 mg, e.g. 50 mg, 75 mg, 100 mg, 125 mg, 150 mg, 175 mg or 200 mg. Normally, a pharmaceutical composition containing 1.1 is administered once or twice daily, a twice daily administration is preferred. Oral administration of 1.1 is preferred.

1.3 is by preference administered subcutaneously. Since 1.1 and 1.3 are different prodrugs of the same active principle (i.e. of 1.2), the dosage of 1.3 is to be adapted to the different administration route in a way that the plasma levels of the active principle will roughly be the same as those obtained by application of the above-mentioned amounts of 1.1.

In a pharmaceutical composition according to the invention, 2a.1 (ASA) may be present in an amount between 50 mg and 500 mg; preferred dosages for 2a.1 are e.g. 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg and 500 mg.

In a pharmaceutical composition according to the invention, 2a.2 (clopidogrel) may be present in an amount between 75 mg and 600 mg; preferred dosages for 2a.2 are e.g. 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg and 600 mg.

The above-mentioned dosages for the compounds 1 and 2a may be combined in any possible way for the binary and ternary combinations.

For instance, the normally recommended dose for the drug may be the dose disclosed in Rote Liste® 2005, Editio Cantor Verlag Aulendorf, Germany, or to Physician's Desk Reference, 58 edition, 2004, e.g. exemplary for melagatran 3 mg/0.3 ml s.c. two times a day, or for ximelagatran 24 mg orally two times a day.

### Formulations and Dosages: ASA

With respect to ASA any of the oral formulations on the market may be used. Reference is made to Rote Liste® 2004, Editio Cantor Verlag Aulendorf, Germany, or to Physician's Desk Reference, 58 edition, 2004. This component of the medication may be administered orally in a daily dosage of 10 to 1000 mg, preferably 25 to 600 mg, e.g. 100 to 300 mg, most preferred 50 to 500 mg, for instance 75 mg twice a day.

### Formulations and Dosages: Clopidogrel

Suitable oral formulations of clopidogrel are disclosed in Rote Liste® 2004, Editio Cantor Verlag Aulendorf, Germany, or in Physician's Desk Reference, 58 edition, 2004, and may contain from 25 mg to 1000 mg, preferably from 75 mg to 600 mg, and most preferably from 75 mg to 400 mg of clopidogrel. For example, the formulation used may contain 25 mg, 50 mg, 75 mg, 150 mg, 250 mg, or 500 mg of clopidogrel. Oral administration may be in one or divided doses of two, three, or four times daily. A single daily dose is preferred. Clopidogrel is on the market under the brand names Plavix® and Iscover®.

### Formulations and Dosages: Ticlopidine

Suitable oral formulations of ticlopidine are disclosed in Rote Liste® 2004, Editio Cantor Verlag Aulendorf, Germany, or in Physician's Desk Reference, 58 edition, 2004, and may contain from 25 mg to 600 mg, preferably from 100 mg to 400 mg, and most preferably from 200 mg to 300 mg of ticlopidine. For example, the formulation may contain 25 mg, 50 mg, 75 mg, 150 mg, 250 mg, or 500 mg of ticlopidine. Oral administration may be in one or divided doses of two, three, or four times daily. A single daily dose is preferred.

It is clear to anyone skilled in the art that the suggested dosages per single dose specified above are not to be regarded as being limited to the numerical values actually stated. Fluctuations of about ±2.5 mg, particularly in the decimal range, are also included, as will be apparent to the skilled man. In these dosage ranges, the active substances 1 and 2a may be present in the weight ratios given above.

For example, without restricting the scope of the invention thereto, the combinations in which the preferred direct thrombin inhibitor 1.1 is used and in which 2a denotes ASA and/or clopidogrel, the pharmaceutical compositions according to the invention may contain for instance the following quantities for each single dose: 150 mg of 1 and 75 mg of clopidogrel and/or 200 mg of ASA.

The dosage of 1.1 may range from 50 to 400 mg/day.

The dosage of 2a.1 may range from 50 to 500 mg/day, preferably from 75 to 325 mg/day.

The dosage of 2a.2 may range from 75 to 600 mg/day.

### Pharmaceutical Compositions Comprising a Direct Thrombin Inhibitor 1 and a Low Molecular Weight Heparin 2b:

One embodiment of the invention is a pharmaceutical composition comprising an direct thrombin inhibitor 1 and a low molecular weight heparins (LMWH) resp. heparinoids resp. unfractionated heparin 2b. Binary compositions containing only one active compound 1 and one active compound 2b, optionally together with one or more pharmaceutically acceptable excipients or carriers, are preferred. In the pharmaceutical combinations according to the invention preferred heparins 2b are selected from the group consisting of enoxaparin, reviparin, dalteparin, tinzaparin, nadroparin and danaparoid.

Suitable doses resp. dose ranges for the active compounds 2b are:

enoxaparin: 40 mg qd, 30 mg bid, 1.5 mg/kg once daily or 1.0 mg/kg twice daily
reviparin: 1750 U/day
dalteparin: 2500-5000 IU/day
tinzaparin: 50-75 IU/kg or 3500 IU/day
nadroparin: 3075 IU/day
danaparoid: 750 IU/day.

Compounds 2b are usually administered parentally, by preference subcutaneously. Furthermore, suitable doses and formulations for compounds 2b are described in Rote Liste® 2005, Editio Cantor Verlag Aulendorf, Germany, or to Physician's Desk Reference, 58 edition, 2004.

The dose ranges of 1 have already been given above.

Preferably, the compound 2b is enoxaparin.

Any reference to steroids 2b within the scope of the present invention includes a reference to the salts or derivatives which may be formed from the heparins. Examples of possible salts or derivatives include: sodium salts, sulphobenzoates, phosphates, isonicotinates, acetates, propionates, dihydrogen phosphates, palmitates, pivalates or furoates. In some cases the compounds of formula 2b may also occur in the form of their hydrates. Any reference to heparins 2b within the scope of the present invention also includes a reference to the compounds 2b in the form of their diastereomers, mixtures of diastereomers or in the form of the racemates.

The proportions in which the active substances 1 and 2b may be used in the active substance combinations according to the invention are variable. Active substances 1 and 2b may possibly be present in the form of their solvates or hydrates. Depending on the choice of the compounds 1 and 2b, the weight ratios which may be used within the scope of the present invention vary on the basis of the different molecular weights of the various compounds and their different potencies.

### Pharmaceutical Compositions Comprising a Direct Thrombin Inhibitor 1 and a Factor Xₐ Inhibitor 2c:

One embodiment of the invention is a pharmaceutical composition comprising an direct thrombin inhibitor 1 and a factor Xₐ inhibitor 2c. Binary compositions containing only one active 1 and one active 2c, optionally together with one or more pharmaceutically acceptable excipients or carriers, are preferred. In the pharmaceutical combinations according to the invention preferred a factor Xₐ inhibitors 2c are selected from the group consisting of
(1) fondaparinux,
(2) idraparinux,
(3) Razaxaban (DPC-906; Curr Hematol Rep. 2004 September; 3(5): 357-62),
(4) Apixaban (BMS-562247)
(5) N-(4-Bromo-2-{[(5-chloropyridin-2-yl)amino]carbonyl}-6-hydroxyphenyl)-1-isopropylpiperidin-4-carboxamid (JP 2005179272)

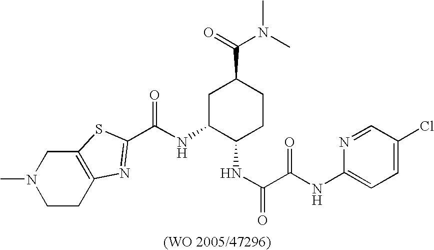

(6)
(10) 5-chloro-N-[((5S)-2-oxo-3-[4-(3-oxo-4-morpholinyl)phenyl]-1,3-oxazolidin-5-yl)methyl]-2-thiophenearboxamide (BAY-59-7939, WO 04/60887)
(11) 1-(indole-6-carbonyl-D-phenylglycinyl)-4-(1-methyl-piperidin-4-yl)piperazine (LY-517717, WO 02/100847)
(12) 2-(5-carbamimidoyl-2-hydroxy-phenyl)-N-[3-methyl-4-(pyrrolidin-1-yl-carbonyl)-phenyl]-acetamide (WO 03/037220)
(13) 2-(3-carbamimidoyl-phenyl)-N-[3-methyl-4-(pyrrolidin-1-yl-carbonyl)-phenyl]-isobutyramide (WO 02/062748)
(14) 2-(5-carbamimidoyl-2-hydroxy-phenyl)-N-[4-(pyrrolidin-1-yl-carbonyl)-3-trifluoromethyl-phenyl]-propionamide (WO 02/062748)
(15) 2-(3-carbamimidoyl-phenyl)-N-[3-bromo-4-(pyrrolidin-1-yl-carbonyl)-phenyl]-3-(pyridin-4-yl)-propionamide (WO 02/062748)
(16) N-(5-carbamimidoyl-2-hydroxy-benzyl)-3-methyl-4-(pyrrolidin-1-yl-carbonyl)-benzamide (WO 02/062778)
(17) ethyl 2-(3-carbamimidoyl-phenyl)-2-[3-methyl-4-(pyrrolidin-1-yl-carbonyl)-benzoylamino]-acetate (WO 02/062778)
(18) (1) N-(5-Amidino-2-hydroxy-benzyl)-3-trifluormethyl-4-(3-aminomethyl-1,4,5,6-tetrahydro-cyclopentapyrazol-1-yl)-benzamide (WO 02/072558)
(19) 6) N-[1-(5-Amidino-2-hydroxy-phenyl)-ethyl]-3-trifluormethyl-4-(4,5,6,7-tetrahydro-benzimidazol-1-yl)-benzamide (WO 02/072558)
(20) N-(5-Amidino-2-hydroxy-benzyl)-3-trifluormethyl-4-(3-methyl-1,4,5,6-tetrahydro-cyclopentapyrazol-1-yl)-benzamide (WO 02/072558)
(21) 2-(5-amidino-2-hydroxy-phenyl)-N-[3-trifluoromethyl-4-(pyrrolidin-1-yl-carbonyl)-phenyl]-3-phenyl-propionamide (WO 04/013115)
(22) 4-hydroxy-3-{[6-chloro-7-(pyrrolidin-1-yl-carbonyl)-quinazolin-4-yl]aminomethyl}-benzamidine (WO 2004/080970)
(23) 4-hydroxy-3-{[7-methoxy-6-(pyrrolidin-1-yl-carbonyl)-isoquinolin-1-yl]aminomethyl}-benzamidine (WO 2004/080970)
(24) 4-hydroxy-3-{2-phenyl-1-[7-(pyrrolidin-1-yl-carbonyl)-quinazolin-4-ylamino]-ethyl}-benzamidine (WO 2004/080970)
(25) 4-hydroxy-3-{[6-methyl-7-(pyrrolidin-1-yl-carbonyl)-quinazolin-4-yl]aminomethyl}-benzamidine (WO 2004/080970)
(26) 4-hydroxy-3-{[7-(pyrrolidin-1-yl-carbonyl)-quinazolin-4-yl]aminomethyl}-benzamidine (WO 2004/080970)
(27) ethyl 3-(3-amidino-phenyl)-3-{[6-chloro-7-(pyrrolidin-1-yl-carbonyl)-quinazolin-4-yl]amino}-propionate (WO 2004/080970)
(28) 3-(3-amidino-phenyl)-3-{[6-chloro-7-(pyrrolidin-1-yl-carbonyl)-quinazolin-4-yl]amino}-propionic acid (WO 2004/080970)
(29) N-benzoyl-4-hydroxy-3-{[7-(pyrrolidin-1-yl-carbonyl)-quinazolin-4-yl]aminomethyl}-benzamidine (WO 2004/080970)
(30) N-hydroxy-4-hydroxy-3-{[6-methyl-7-(pyrrolidin-1-yl-carbonyl)-quinazolin-4-yl]aminomethyl}-benzamidine (WO 2004/080970)
(31) N-acetoxymethoxycarbonyl-4-hydroxy-3-{[6-methyl-7-(pyrrolidin-1-yl-carbonyl)-quinazolin-4-yl]aminomethyl}-benzamidine (WO 2004/080970)
   their stereoisomers such as enantiomers and diastereomers, mixtures of stereoisomers such as racemates, prodrugs, pharmacologically acceptable salts, solvates, e.g. hydrates, and physical modifications thereof, e.g. polymorphs.

Prodrugs of the drugs mentioned above are such derivatives containing one or more groups capable of being cleaved in vivo, particularly a group which can be converted in vivo into a carboxy group or/and a group capable of being cleaved in vivo from an imino or amino group. Compounds containing two groups capable of being cleaved in vivo are so-called double prodrugs. Groups which can be converted in vivo into a carboxy group and groups capable of being cleaved in vivo from an imino or amino group are disclosed e.g. in WO 98/37075, being herewith incorporated by reference, as well as in other WO publications cited hereinbefore in connection with specific antithrombotics.

The dose of fondaparinux is of about 2.5 mg/kg/day. Both fondaparinux and idraparinux are by preference administered subcutaneously.

The dose ranges of 1 have already been given above.

Pharmaceutically acceptable salt forms of the active compounds within the pharmaceutical composition of the present invention are prepared for the most part by conventional means. Where the component compound contains a carboxylic acid group, a suitable salt thereof may be formed by reacting the compound with an appropriate base to provide the corresponding base addition salt. Examples of such bases are alkali metal hydroxides including potassium hydroxide, sodium hydroxide, and lithium hydroxide; alkaline earth metal hydroxides such as barium hydroxide and calcium hydroxide; alkali metal alkoxides, e.g., potassium ethanolate and sodium propanolate; and various organic bases such as piperidine, diethanolamine, and N-methylglutamine. Also included are the aluminum salts of the component compounds of the present invention.

For certain component compounds acid addition salts may be formed by treating said compounds with pharmaceutically acceptable organic and inorganic acids, e.g., hydrohalides such as hydrochloride, hydrobromide, hydroiodide; other mineral acids and their corresponding salts such as sulfate, nitrate, phosphate, etc.; and alkyl- and mono-arylsulfonates such as ethanesulfonate, toluenesulfonate, and benzenesulfonate; and other organic acids and their corresponding salts such as acetate, tartrate, maleate, succinate, citrate, benzoate, salicylate, ascorbate, etc.

Accordingly, the pharmaceutically acceptable acid addition salts of the component compounds of the present invention include, but are not limited to: acetate, adipate, alginate, arginate, aspartate, benzoate, benzenesulfonate (besylate), bisulfate, bisulfate, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, phthalate.

Particularly preferred examples of pharmacologically acceptable acid addition salts of the compounds 2c according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid, 1-hydroxy-2-naphthalenecarboxylic acid or maleic acid. If desired, mixtures of the abovementioned acids may also be used to prepare the salts 2c.

In the pharmaceutical compositions according to the invention, the compounds 2c may be present in the form of their racemates, enantiomers or mixtures thereof. The separation of the enantiomers from the racemates may be carried out using methods known in the art (e.g. by chromatography on chiral phases, etc.).

The proportions in which the active substances 1 and 2c may be used in the active substance combinations according to the invention are variable. Active substances 1 and 2c may possibly be present in the form of their solvates or hydrates. Depending on the choice of the compounds 1 and 2c, the weight ratios which may be used within the scope of the present invention vary on the basis of the different molecular weights of the various salt forms.

### Pharmaceutical Compositions Comprising a Direct Thrombin Inhibitor 1 and a Combined Thrombin/Factor Xₐ Inhibitor 2d:

One embodiment of the invention is a pharmaceutical composition comprising an direct thrombin inhibitor 1 and a combined thrombin/factor Xₐ inhibitor 2d. Binary compositions containing only one active compound 1 and one active compound 2d, optionally together with one or more pharmaceutically acceptable excipients or carriers, are preferred.

Combined thrombin/factor Xₐ inhibitors applicable within the scope of the invention are known in the art. Within the scope of the present invention the term combined thrombin/factor Xₐ inhibitors 2d denotes compounds selected from the compounds:
(32) 1-methyl-2-[N-(4-amidinophenyl)-aminomethyl]-5-[N-(hydroxycarbonylmethyl)-quinoline-8-sulphonylamino]-benzimidazole (U.S. Pat. No. 6,121,308)
(33) (R)-2-(4-amidinophenylaminomethyl)-1-methyl-5-[1-(carboxymethylamino)-1-(pyrrolidinocarbonyl)-ethyl]-benzimidazole (WO 00/01704)
(34) 2-(4-amidinophenylaminomethyl)-1-methyl-5-[1-(carboxymethylaminomethyl)-1-(pyrrolidinocarbonyl)-ethyl]-benzimidazole (WO 01/47896)
(35) (R)-2-[4-(N-phenylcarbonylamidino)-phenylaminomethyl]-1-methyl-5-[1-(n-propyloxycarbonylmethylamino)-1-(pyrrolidinocarbonyl)-ethyl]-benzimidazole (WO 01/47896)
(36) 3-{[6-(N-acetyl-N-cyclopentylamino)-7-methyl-isoquinolin-1-yl]aminomethyl}-4-hydroxy-benzamidine (WO 2004/080970)
   (the following compounds are disclosed in WO 2004/056784)
(37) N-[1-(5-chloro-1H-benzimidazol-2-yl)-ethyl]-3-methyl-4-(2,5-dihydro-pyrrol-1-yl-carbonyl)-benzamide
(38) N-[1-(5-chloro-1H-benzimidazol-2-yl)-ethyl]-3-ethyl-4-(pyrrolidin-1-yl-carbonyl)-benzamide
(39) N-[1-(5-chloro-1H-benzimidazol-2-yl)-ethyl]-3-chloro-4-(2-aminomethyl-pyrrolidin-1-yl-carbonyl)-benzamide
(40) 3-chloro-N-(5-chloro-1H-benzimidazol-2-yl-methyl)-4-(3-oxo-piperazin-1-yl-carbonyl)-benzamide
(41) N-[1-(5-bromo-1H-benzimidazol-2-yl)-ethyl]-3-methyl-4-(pyrrolidin-1-yl-carbonyl)-benzamide
(42) N-[(5-chloro-1H-benzimidazol-2-yl)-phenyl-methyl]-3-methyl-4-(pyrrolidin-1-yl-carbonyl)-benzamide
(43) N-[1-(5-chloro-1H-benzimidazol-2-yl)-3-methyl-butyl]-3-methyl-4-(pyrrolidin-1-yl-carbonyl)-benzamide
(44) (S)—N-[1-(5-chloro-1H-benzimidazol-2-yl)]ethyl-3-methyl-4-(pyrrolidin-1-yl-carbonyl)-benzamide
(45) N-[(1S)-1-(5-chloro-1H-benzimidazol-2-yl)-ethyl]-3-chloro-4-[(2R/S)-2-amino-methyl-pyrrolidin-1-yl-carbonyl)-benzamide
(46) N-[(1S)-1-(5-chloro-1H-benzimidazol-2-yl)-3-methylsulphanyl-propyl]-3-chloro-4-[(2S)-2-(N-tert.-butoxycarbonyl-aminomethyl)-pyrrolidin-1-yl-carbonyl]-benzamide
(47) N-[(1S)-1-(5-chloro-1H-benzimidazol-2-yl)-butyl]-3-chloro-4-[(2S)-2-amino-methyl-pyrrolidin-1-yl-carbonyl]-benzamide
(48) N-[(1S)-1-(5-chloro-1H-benzimidazol-2-yl)-3-methylsulphanyl-propyl]-3-chloro-4-[(2S)-2-aminomethyl-pyrrolidin-1-yl-carbonyl]-benzamide
(49) N-[(1S)-1-(5-chloro-1H-benzimidazol-2-yl)-3-methylsulphinyl-propyl]-3-chloro-4-[(2S)-2-aminomethyl-pyrrolidin-1-yl-carbonyl]-benzamide
(50) N-[(1S)-1-(5-chloro-1H-benzimidazol-2-yl)-3-methylsulphonyl-propyl]-3-chloro-4-[(2S)-2-aminomethyl-pyrrolidin-1-yl-carbonyl]-benzamide
(51) N-[(1S)-5-(benzyloxycarbonylamino)-1-(5-chloro-1H-benzimidazol-2-yl)-pentyl]-3-methyl-4-(pyrrolidin-1-yl-carbonyl)-benzamide
(52) N-[(1S)-1-(5-chloro-1H-benzimidazol-2-yl)-3-phenyl-propyl]-3-methyl-4-(pyrrolidin-1-yl-carbonyl)-benzamide
(53) N-[(1S)-1-(5-chloro-1H-benzimidazol-2-yl)-3-methylsulphanyl-propyl]-3-methyl-4-(pyrrolidin-1-yl-carbonyl)-benzamide
(54) N-[(1S)-3-benzyloxycarbonyl-1-(5-chloro-1H-benzimidazol-2-yl)-propyl]-3-methyl-4-(pyrrolidin-1-yl-carbonyl)-benzamide
(55) N-[(1S)-1-(5-chloro-1H-benzimidazol-2-yl)-3-(pyrrolidin-1-yl-carbonyl)-propyl]-3-methyl-4-(pyrrolidin-1-yl-carbonyl)-benzamide
(56) N-[(1R)-1-(5-chloro-1H-benzimidazol-2-yl)-2-hydroxy-ethyl]-3-methyl-4-(pyrrolidin-1-yl-carbonyl)-benzamide
(57) N -[1-(5-chloro-1H-benzimidazol-2-yl)-2-methoxy-ethyl]-3-methyl-4-(pyrrolidin-1-yl-carbonyl)-benzamide
(58) N-[(1S)-1-(5-chloro-1H-benzimidazol-2-yl)-3-methoxy-propyl]-3-methyl-4-(pyrrolidin-1-yl-carbonyl)-benzamide
(59) N-[(1R)-2-(C-tert.butoxycarbonyl-methyloxy)-1-(5-chloro-1H-benzimidazol-2-yl)-ethyl]-3-methyl-4-(pyrrolidin-1-yl-carbonyl)-benzamide
(60) N-[(1S)-1-(5-chloro-1H-benzimidazol-2-yl)-3-methylsulphinyl-propyl]-3-methyl-(pyrrolidin-1-yl-carbonyl)-benzamide
(61) N-[(5-chloro-1H-benzimidazol-2-yl)-phenyl-methyl]-3-methyl-4-(pyrrolidin-1-yl-carbonyl)-benzamide
(62) N-[1-(5-chloro-1H-benzimidazol-2-yl)-phenyl-methyl]-4-(2,5-dihydro-pyrrol-1-yl-carbonyl)-3-methyl-benzamide
(63) N-[(1S)-1-(5-chloro-1H-benzimidazol-2-yl)-3-methylsulphonylamino-propyl]-3-methyl-4-(pyrrolidin-1-yl-carbonyl)-benzamide
(64) N-{(1S)-1-(5-chloro-1H-benzimidazol-2-yl)-3-[3-(2-chloro-ethyl)-ureido]-propyl}-3-methyl-4-(pyrrolidin-1-yl-carbonyl)-benzamide
(65) N-[(1S)-1-(5-chloro-1H-benzimidazol-2-yl)-butyl]-3-methyl-4-(pyrrolidin-1-yl-carbonyl)-benzamide
(66) 3-bromo-N-[(1S)-1-(5-chloro-1H-benzimidazol-2-yl)-3-methylsulphanyl-propyl]-4-(pyrrolidin-1-yl-carbonyl)-benzamide
(67) 3-chloro-N-[(1S)-1-(5-chloro-1H-benzimidazol-2-yl)-3-(methylsulphanyl)-propyl]-4-(pyrrolidin-1-yl-carbonyl)-benzamide
(68) 3-bromo-N-[(1S)-1-(5-chloro-1H-benzimidazol-2-yl)-3-(methylsulphonyl)-propyl]-4-(pyrrolidin-1-yl-carbonyl)-benzamide
(69) 3-bromo-N-[(1S)-1-(5-chloro-1H-benzimidazol-2-yl)-3-methylsulphinyl-propyl]-4-(pyrrolidin-1-yl-carbonyl)-benzamide
(70) 3-chloro-N-[(1S)-1-(5-chloro-1H-benzimidazol-2-yl)-ethyl]-4-[(2R)-2-(methyl-sulphonylamino-methyl)-pyrrolidin-1-yl-carbonyl]-benzamide
(71) (1R)-3-bromo-N-[1-(5-chloro-1H-benzimidazol-2-yl)-2-hydroxy-ethyl]-4-(2,5-dihydro-pyrrol-1-yl-carbonyl)-benzamide
(72) (1R)-3-methyl-N-[1-(5-chloro-1H-benzimidazol-2-yl)-2-methoxy-ethyl]-4-(2,5-dihydro-pyrrol-1-yl-carbonyl)-benzamide
(73) (1R)-3-chloro-N-[1-(5-chloro-1H-benzimidazol-2-yl)-2-hydroxy-ethyl]-4-(2,5-dihydro-pyrrol-1-yl-carbonyl)-benzamide
(74) N-{(1S)-1-(5-chloro-1H-benzimidazol-2-yl)-3-[(3R,S)-3-dimethylamino-pyrrolidin-1-yl]-carbonyl-propyl}-3-methyl-4-(pyrrolidin-1-yl-carbonyl)-benzamide
(75) N-{(1S)-1-(5-chloro-1H-benzimidazol-2-yl)-3-[(2R)-2-hydroxymethyl-pyrrolidin-1-yl-carbonyl]-propyl}-3-methyl-4-(pyrrolidin-1-yl-carbonyl)-benzamide
(76) N-{(1S)-1-(5-chloro-1H-benzimidazol-2-yl)-3-[(2S)-2-hydroxymethyl-pyrrolidin-1-yl-carbonyl]-propyl}-3-methyl-4-(pyrrolidin-1-yl-carbonyl)-benzamide
(77) N-[(1S)-1-(5-chloro-1H-benzimidazol-2-yl)-3-(2-methyl-2,6-diaza-spiro[3.4]oct-6-yl-carbonyl)-propyl]-3-methyl-4-(pyrrolidin-1-yl-carbonyl)-benzamide
(78) N-{(1S)-3-[(1R)-2-(aminocarbonyl)-pyrrolidin-1-yl-carbonyl]-1-(5-chloro-1H-benzimidazol-2-yl)-propyl}-3-methyl-4-(pyrrolidin-1-yl-carbonyl)-benzamide
(79) N-{(1S)-1-(5-chloro-1H-benzimidazol-2-yl)-3-[(2R)-2-tert.butoxycarbonyl-aminomethyl-pyrrolidin-1-yl-carbonyl]-propyl}-3-methyl-4-(pyrrolidin-1-yl-carbonyl)-benzamide
(80) N-{(1S)-1-(5-chloro-1H-benzimidazol-2-yl)-3-[(3R,S)-hydroxymethyl-pyrrolidin-1-yl)-carbonyl]-propyl}-3-methyl-4-(pyrrolidin-1-yl-carbonyl)-benzamide
(81) N-[(1S)-1-(5-chloro-1H-benzimidazol-2-yl)-3-(1,1-dioxo-1-thiomorpholine-4-yl-carbonyl]-propyl]-3-methyl-4-(pyrrolidin-1-yl-carbonyl)-benzamide
(82) N-[(1S)-1-(5-chloro-1H-benzimidazol-2-yl)-3-[(4-methyl-3-oxo-piperazin-1-yl-carbonyl)-propyl]-3-methyl-4-(pyrrolidin-1-yl-carbonyl)-benzamide
(83) N-[(1R)-1-(5-chloro-1H-benzimidazol-2-yl)-2-methoxy-ethyl]-3-methyl-4-(pyrrolidin-1-yl-carbonyl)-benzamide
(84) 3-chloro-N-[(1R)-1-(5-chloro-1H-benzimidazol-2-yl)-2-methoxy-ethyl]-4-(2,5-dihydro-pyrrol-1-yl-carbonyl)-benzamide
(85) 3-bromo-N-[(1R)-1-(5-chloro-1H-benzimidazol-2-yl)-2-methoxy-ethyl]-4-(pyrrolidin-1-yl-carbonyl)-benzamide
(86) 3-bromo-N-[(1R)-1-(5-chloro-1H-benzimidazol-2-yl)-2-methoxy-ethyl]-4-(2,5-dihydro-pyrrol-1-yl-carbonyl)-benzamide
(87) 3-methyl-N-[(1R)-1-(5-chloro-1H-benzimidazol-2-yl)-2-hydroxy-ethyl]-4-(2,5-dihydro-pyrrol-1-yl-carbonyl)-benzamide
(88) N-{(1S)-1-(5-chloro-1H-benzimidazol-2-yl)-3-[(25)-2-aminomethyl-pyrrolidin-1-yl-carbonyl]-propyl}-3-methyl-4-(pyrrolidin-1-yl-carbonyl)-benzamide
(89) N-{(1S)-1-(5-chloro-1H-benzimidazol-2-yl)-3-[(2R)-2-aminomethyl-pyrrolidin-1-yl-carbonyl]-propyl}-3-methyl-4-(pyrrolidin-1-yl-carbonyl)-benzamide
(90) 3-chloro-N-[(1R,S)-1-(5-chloro-1H-benzimidazol-2-yl)-ethyl]-4-[(2R)-2-methoxy-methyl-pyrrolidin-1-yl-carbonyl]-benzamide
(91) 3-chloro-N-[1-(5-chloro-1H-benzimidazol-2-yl)-ethyl]-4-(3,4,5,6-tetrahydro-2H-[2,3]-bipyridinyl-1-yl-carbonyl)-benzamide
(92) N-[(1R)-1-(5-chloro-1H-benzimidazol-2-yl)-2-methoxy-ethyl]-4-(pyrrolidin-1-yl-carbonyl)-3-trifluoromethyl-benzamide
(93) N-[(1S)-1,3-bis-(5-chloro-1H-benzimidazol-2-yl)-propyl]-3-methyl-4-(pyrrolidin-1-yl-carbonyl)-benzamide
(94) 3-chloro-N-[(1S)-1-(5-chloro-1H-benzimidazol-2-yl)-ethyl]-4-[(2R/S)-2-dimethyl-aminomethyl-pyrrolidin-1-yl-carbonyl]-benzamide
(95) N-[(1S)-1-(5-chloro-1H-benzimidazol-2-yl)-3-methanesulphonylamino-propyl]-4-(2,5-dihydro-pyrrol-1-yl-carbonyl)-3-methyl-benzamide
(96) N-[(1S)-1-(5-chloro-1H-benzimidazol-2-yl)-butyl]-4-(2,5-dihydro-pyrrol-1-yl-carbonyl)-3-methyl-benzamide
(97) 3-chloro-N-[(1S)-1-(5-chloro-1H-benzimidazol-2-yl)-butyl]-4-(2,5-dihydro-pyrrol-1-yl-carbonyl)-benzamide
(98) 3-bromo-N-[(1S)-1-(5-chloro-1H-benzimidazol-2-yl)-butyl]-4-(2,5-dihydro-pyrrol-1-yl-carbonyl)-benzamide
(99) 4-(N-acetyl-N-cyclopentyl-amino)-N-[(1S)-1-(5-chloro-1H-benzimidazol-2-yl)-2-methylsulphanyl-ethyl]-3-methyl-benzamide
(100) 3-chloro-N-[(1S)-1-(5-chloro-1H-benzimidazol-2-yl)-ethyl]-4-[(2R)-2-(pyrrolidin-1-yl-methyl)-pyrrolidin-1-yl-carbonyl]-benzamide
(101) 3-bromo-N-[(1R)-1-(5-bromo-1H-benzimidazol-2-yl)-2-methoxy-ethyl]-4-(2,5-dihydro-pyrrol-1-yl-carbonyl)-benzamide
(102) 3-bromo-N-[(1R)-1-(5-chloro-1H-benzimidazol-2-yl)-2-ethoxy-ethyl]-4-(2,5-dihydro-pyrrol-1-yl-carbonyl)-benzamide
(103) N-[(1R)-2-allyloxy-1-(5-chloro-1H-benzimidazol-2-yl)-ethyl]-4-(2,5-dihydro-pyrrol-1-yl-carbonyl)-3-methyl-benzamide
(104) 3-bromo-N-[(1R)-1-(5-chloro-1H-benzimidazol-2-yl)-2-prop-2-ynyloxy-ethyl]-4-(2,5-dihydro-pyrrol-1-yl-carbonyl)-benzamide
(105) N-[(1S)-1-(5-chloro-1H-benzimidazol-2-yl)-3-(1H-tetrazol-5-yl)-propyl]-3-methyl-4-(pyrrolidin-1-yl-carbonyl)-benzamide
(106) N-[(1R)-1-(5-chloro-1H-benzimidazol-2-yl)-2-hydroxy-ethyl]-4-(2,5-dihydro-pyrrol-1-yl-carbonyl)-3-trifluoromethyl-benzamide
(107) 3-chloro-N-[(1R)-1-(5-bromo-1H-benzimidazol-2-yl)-2-hydroxy-ethyl]-4-(2,5-dihydro-pyrrol-1-yl-carbonyl)-benzamide
(108) 3-bromo-N-[(1R)-1-(5-bromo-1H-benzimidazol-2-yl)-2-hydroxy-ethyl]-4-(pyrrolidin-1-yl-carbonyl)-benzamide
(109) 3-methyl-N-[(1R)-1-(5-bromo-1H-benzimidazol-2-yl)-2-hydroxy-ethyl]-4-(pyrrolidin-1-yl-carbonyl)-benzamide
   (the following compounds are disclosed in WO 2004-058743)
(110) 6-chloro-4-[1-(5-chloro-1H-benzimidazol-2-yl)-ethylamino]-7-(2-aminomethyl-pyrrolidin-1-yl-carbonyl)-quinazoline
(111) 6-chloro-4-[1-(S)-(5-chloro-1H-benzimidazol-2-yl)-ethylamino]-7-(2,5-dihydro-pyrrol-1-yl-carbonyl)-quinazoline
(112) 6-chloro-4-[1-(S)-(5-chloro-1H-benzimidazol-2-yl)-ethylamino]-7-(pyrrolidin-1-yl-carbonyl)-quinazoline
(113) 4-[1-(5-chloro-1H-benzimidazol-2-yl)-3-methylsulphanyl-propylamino]-6-methyl-7-(pyrrolidin-1-yl-carbonyl)-quinoline
(114) 4-[1-(5-chloro-1H-benzimidazol-2-yl)-ethylamino]-6-methyl-7-(pyrrolidin-1-yl-carbonyl)-quinoline
(115) 4-[1-(5-chloro-1H-benzimidazol-2-yl)-ethylamino]-6-methyl-7-(3-oxo-piperazin-1-yl-carbonyl)-quinoline
(116) 4-[(1R/S)-1-(5-chloro-1H-benzimidazol-2-yl)-ethylamino]-6-methyl-7-[(2R)-2-aminomethyl-pyrrolidin-1-yl-carbonyl]-quinoline
(117) 4-[1-(5-chloro-1H-benzimidazol-2-yl)-3-methylsulphanyl-propylamino]-6-methyl-7-(3-oxo-piperazin-1-yl-carbonyl)-quinoline
(118) 4-[1-(5-chloro-1H-benzimidazol-2-yl)-3-methanesulphonyl-propylamino]-6-methyl-7-(pyrrolidin-1-yl-carbonyl)-quinoline
(119) 6-chloro-4-[(1S)-1-(5-chloro-1H-benzimidazol-2-yl)-ethylamino]-7-[(2R)-2-aminomethyl-pyrrolidin-1-yl-carbonyl]-quinazoline
(120) 6-chloro-4-[(1S)-1-(5-chloro-1H-benzimidazol-2-yl)-2-hydroxy-ethylamino]-7-(2,5-dihydropyrrol-1-yl-carbonyl)-quinazoline
(121) 6-chloro-4-[(1S)-1-(5-chloro-1H-benzimidazol-2-yl)-2-hydroxy-ethylamino]-7-[(2R)-2-aminomethyl-pyrrolidin-1-yl-carbonyl]-quinazoline
(122) 6-chloro-4-[1-(5-chloro-1H-benzimidazol-2-yl)-3-hydroxycarbonylpropylamino]-7-(pyrrolidin-1-yl-carbonyl)-quinazoline
(123) 6-chloro-4-[1-(5-chloro-1H-benzimidazol-2-yl)-3-benzyloxycarbonylpropyl-amino]-7-(pyrrolidin-1-yl-carbonyl)-quinazoline
(124) 6-chloro-4-[(1S)-1-(5-chloro-1H-benzimidazol-2-yl)-3-methylsulphanyl-propyl-amino]-7-[(2R)-2-tert.-butyloxycarbonyl-aminomethyl-pyrrolidin-1-yl-carbonyl]-quinazoline
(125) 6-chloro-4-[(1S)-1-(5-chloro-1H-benzimidazol-2-yl)-3-methylsulphanyl-propyl-amino]-7-(pyrrolidin-1-yl-carbonyl)-quinazoline
(126) 6-chloro-4-[(1S)-1-(5-chloro-1H-benzimidazol-2-yl)-3-methoxy-propylamino]-7-(2,5-dihydropyrrol-1-yl-carbonyl)-quinazoline
(127) 6-chloro-4-[(1S)-1-(5-chloro-1H-benzimidazol-2-yl)-3-methanesulphonyl-propyl-amino]-7-(pyrrolidin-1-yl-carbonyl)-quinazoline
(128) 6-chloro-4-[(1S)-1-(5-chloro-1H-benzimidazol-2-yl)-3-methylsulphanyl-propyl-amino]-7-[(2R)-2-aminomethyl-pyrrolidin-1-yl-carbonyl]-quinazoline
(129) 6-chloro-4-[(1S)-1-(5-chloro-1H-benzimidazol-2-yl)-3-methanesulphinyl-propyl-amino]-7-(pyrrolidin-1-yl-carbonyl)-quinazoline
(130) 6-chloro-4-[(1S)-1-(5-chloro-1H-benzimidazol-2-yl)-3-benzyloxycarbonylpropyl-amino]-7-(pyrrolidin-1-yl-carbonyl)-quinazoline
(131) 6-chloro-4-[(1S)-1-(5-chloro-1H-benzimidazol-2-yl)-2-hydroxy-ethylamino]-7-(piperazin-3-on-1-yl-carbonyl)-quinazoline
(132) 6-chloro-4-[(1S)-1-(5-chloro-1H-benzimidazol-2-yl)-3-hydroxycarbonylpropyl-amino]-7-[(2S)-2-aminomethyl-pyrrolidin-1-yl-carbonyl]-quinazoline
(133) 6-chloro-4-[(1S)-1-(5-chloro-1H-benzimidazol-2-yl)-3-methanesulphonyl-propyl-amino]-7-[(2R)-2-tert.-butyloxycarbonyl-aminomethyl-pyrrolidin-1-yl-carbonyl]-quinazoline
(134) 6-chloro-4-[(1S)-1-(5-chloro-1H-benzimidazol-2-yl)-3-methanesulphonyl-propylamino]-7-[(2R)-2-aminomethyl-pyrrolidin-1-yl-carbonyl]-quinazoline
(135) 6-chloro-4-[(1S)-1-(5-chloro-1H-benzimidazol-2-yl)-ethylamino]-7-(thiazolidin-3-yl-carbonyl)-quinazoline
(136) 6-chloro-4-[(1S)-1-(5-chloro-1H-benzimidazol-2-yl)-3-ethoxycarbonylpropyl-amino]-7-(pyrrolidin-1-yl-carbonyl)-quinazoline
(137) 4-[(1S)-1-(5-chloro-1H-benzimidazol-2-yl)-ethylamino]-6-methyl-7-(pyrrolidin-1-yl-carbonyl)-quinazoline
(138) 4-[(1S)-1-(5-chloro-1H-benzimidazol-2-yl)-ethylamino]-6-methyl-7-(pyrrolidin-1-yl-carbonyl)-quinazoline
(139) 6-chloro-4-[(1S)-1-(5-chloro-1H-benzimidazol-2-yl)-3-methanesulphinyl-propyl-amino]-7-[(2R)-2-aminomethyl-pyrrolidin-1-yl-carbonyl]-quinazoline
(140) 4-[(1S)-1-(5-chloro-1H-benzimidazol-2-yl)-3-methylsulphanyl-propylamino]-6-methyl-7-(2,5-dihydropyrrol-1-yl-carbonyl)-quinazoline
(141) 6-bromo-4-[(1S)-1-(5-chloro-1H-benzimidazol-2-yl)-ethylamino]-7-(2,5-dihydro-pyrrol-1-yl-carbonyl)-quinazoline
(142) 6-chloro-4-[(1S)-1-(5-chloro-1H-benzimidazol-2-yl)-3-ethoxycarbonylpropyl-amino]-7-(2,5-dihydropyrrol-1-yl-carbonyl)-quinazoline
(143) 6-chloro-4-[(1S)-1-(5-chloro-1H-benzimidazol-2-yl)-3-methylsulphanyl-propylamino]-7-(2,5-dihydropyrrol-1-yl-carbonyl)-quinazoline
(144) 6-chloro-4-[(1S)-1-(5-chloro-1H-benzimidazol-2-yl)-butylamino]-7-(2,5-dihydro-pyrrol-1-yl-carbonyl)-quinazoline
(145) 6-chloro-4-[(1S)-1-(5-chloro-1H-benzimidazol-2-yl)-3-methylsulphanyl-propyl-amino]-7-(2,5-dihydropyrrol-1-yl-carbonyl)-quinazoline
(146) 6-chloro-4-[(1S)-1-(5-chloro-1H-benzimidazol-2-yl)-2-methoxy-ethylamino]-7-(2,5-dihydropyrrol-1-yl-carbonyl)-quinazoline
(147) 6-chloro-4-[1-(5-chloro-1H-benzimidazol-2-yl)-3-diethylaminocarbonyl-propyl-amino]-7-(pyrrolidin-1-yl-carbonyl)-quinazoline
(148) 6-chloro-4-[1-(5-chloro-1H-benzimidazol-2-yl)-3-[N-methyl-N-piperidin-4-yl-amino]-carbonyl-propyl-amino]-7-(pyrrolidin-1-yl-carbonyl)-quinazoline
(149) 6-chloro-4-[1-(5-chloro-1H-benzimidazol-2-yl)-3-[4-methyl-piperazin-1-yl]-carbonyl-propyl-amino]-7-(pyrrolidin-1-yl-carbonyl)-quinazoline
(150) 6-chloro-4-[1-(5-chloro-1H-benzimidazol-2-yl)-3-(C-piperidin-4-yl-methyl-amino)-carbonyl-propyl-amino]-7-(pyrrolidin-1-yl-carbonyl)-quinazoline
(151) 6-chloro-4-[1-(5-chloro-1H-benzimidazol-2-yl)-3-(N-benzyl-N-methyl-amino)-carbonyl-propyl-amino]-7-(pyrrolidin-1-yl-carbonyl)-quinazoline
(152) 4-[(1S)-1-(5-chloro-1H-benzimidazol-2-yl)-3-allyloxycarbonylpropyl-amino]-6-methyl-7-(2,5-dihydropyrrol-1-yl-carbonyl)-quinazoline
(153) 6-bromo-4-[(1S)-1-(5-chloro-1H-benzimidazol-2-yl)-3-allyloxycarbonylpropyl-amino]-7-(2,5-dihydropyrrol-1-yl-carbonyl)-quinazoline
(154) 6-chloro-4-[(1S)-1-(5-chloro-1H-benzimidazol-2-yl)-2-methoxy-ethylamino]-7-(pyrrolidin-1-yl-carbonyl)-quinazoline
(155) 6-chloro-4-[(1S)-1-(5-chloro-1H-benzimidazol-2-yl)-3-methanesulphonyl-propylamino]-1-oxy-7-[(2R)-2-aminomethyl-pyrrolidin-1-yl-carbonyl]-quinazoline
(156) 6-chloro-4-[(1S)-1-(5-chloro-1H-benzimidazol-2-yl)-ethylamino]-7-[(2S)-2-(pyrrolidin-1-yl-methyl)-pyrrolidin-1-yl-carbonyl]-quinazoline
(157) 6-chloro-4-[(1S)-1-(5-chloro-1H-benzimidazol-2-yl)-ethylamino]-7-[(2R/S)-2-aminomethyl-thiazolidinyl-carbonyl]-quinazoline
(158) 6-chloro-4-[(1S)-1-(5-chloro-1H-benzimidazol-2-yl)-3-methanesulphonyl-propylamino]-7-[(2R)-2-(methanesulphonyl-aminomethyl)-pyrrolidin-1-yl-carbonyl]-quinazoline
(159) 6-chloro-4-{1-(5-chloro-1H-benzimidazol-2-yl)-3-[(1,2,3,4-tetrahydroisoquinolin-1-yl)-carbonyl-propyl-amino]}-7-(pyrrolidin-1-yl-carbonyl)-quinazoline
(160) 6-chloro-4-[1-(5-chloro-1H-benzimidazol-2-yl)-3-(benzylamino-carbonyl)-propyl-amino]-7-(pyrrolidin-1-yl-carbonyl)-quinazoline
(161) 6-chloro-4-{1-(5-chloro-1H-benzimidazol-2-yl)-3-[(N-methyl-N-phenethyl-amino-carbonyl)-propyl-amino]}-7-(pyrrolidin-1-yl-carbonyl)-quinazoline
(162) 6-chloro-4-[1-(5-chloro-1H-benzimidazol-2-yl)-3-(hydroxyethylamino-carbonyl)-propyl-amino]-7-(pyrrolidin-1-yl-carbonyl)-quinazoline
(163) 6-chloro-4-{1-(5-chloro-1H-benzimidazol-2-yl)-3-[(C-pyridin-3-yl-methylamino-carbonyl)-propyl-amino]}-7-(pyrrolidin-1-yl-carbonyl)-quinazoline
(164) 6-chloro-4-{1-(5-chloro-1H-benzimidazol-2-yl)-3-[(1-oxa-3,8-diaza-spiro[4.5]decan-2-on-8-yl)-carbonyl]-propyl-amino}-7-(pyrrolidin-1-yl-carbonyl)-quinazoline
(165) 6-chloro-4-[1-(5-chloro-1H-benzimidazol-2-yl)-3-(morpholin-4-yl-carbonyl)-propyl-amino]-7-(pyrrolidin-1-yl-carbonyl)-quinazoline
(166) 6-chloro-4-[1-(5-chloro-1H-benzimidazol-2-yl)-3-(C-cyclohexyl-methylamino-carbonyl)-propyl-amino]-7-(pyrrolidin-1-yl-carbonyl)-quinazoline
(167) 6-chloro-4-[1-(5-chloro-1H-benzimidazol-2-yl)-3-(methoxyethylamino-carbonyl)-propyl-amino]-7-(pyrrolidin-1-yl-carbonyl)-quinazoline
(168) 6-chloro-4-[1-(5-chloro-1H-benzimidazol-2-yl)-3-(dimethylaminoethyl-amino-carbonyl)-propyl-amino]-7-(pyrrolidin-1-yl-carbonyl)-quinazoline
(169) 6-chloro-4-[1-(5-chloro-1H-benzimidazol-2-yl)-3-(cyclopropylamino-carbonyl)-propyl-amino]-7-(pyrrolidin-1-yl-carbonyl)-quinazoline
(170) 6-chloro-4-{(1R/S)-1-(5-chloro-1H-benzimidazol-2-yl)-3-[C-(2R/S)-tetrahydro-furan-2-yl-methylamino-carbonyl)-propyl-amino]}-7-(pyrrolidin-1-yl-carbonyl)-quinazoline
(171) 6-chloro-4-[1-(5-chloro-1H-benzimidazol-2-yl)-3-(dimethylaminopropylamino-carbonyl)-propyl-amino]-7-(pyrrolidin-1-yl-carbonyl)-quinazoline
(172) 6-chloro-4-[1-(5-chloro-1H-benzimidazol-2-yl)-3-(aminoethylamino-carbonyl)-propyl-amino]-7-(pyrrolidin-1-yl-carbonyl)-quinazoline
(173) 6-chloro-4-[1-(5-chloro-1H-benzimidazol-2-yl)-3-(2,2,2-trifluoroethylamino-carbonyl)-propyl-amino]-7-(pyrrolidin-1-yl-carbonyl)-quinazoline
(174) 6-chloro-4-{1-(5-chloro-1H-benzimidazol-2-yl)-3-[N-(2-dimethylamino-ethyl)-N-methyl-amino-carbonyl]-propyl-amino}-7-(pyrrolidin-1-yl-carbonyl)-quinazoline
(175) 6-chloro-4-[1-(5-chloro-1H-benzimidazol-2-yl)-3-(N-piperidin-2-yl-amino-carbonyl)-propyl-amino]-7-(pyrrolidin-1-yl-carbonyl)-quinazoline
(176) 6-chloro-4-{1-(5-chloro-1H-benzimidazol-2-yl)-3-[C-(tetrahydropyran-4-yl)-methylamino-carbonyl]-propyl-amino}-7-(pyrrolidin-1-yl-carbonyl)-quinazoline
(177) 6-chloro-4-[1-(5-chloro-1H-benzimidazol-2-yl)-3-(4-hydroxypiperidin-1-yl-carbonyl)-propyl-amino]-7-(pyrrolidin-1-yl-carbonyl)-quinazoline
(178) 6-chloro-4-{1-(5-chloro-1H-benzimidazol-2-yl)-3-[C-(pyridin-4-yl)-methylamino-carbonyl]-propyl-amino}-7-(pyrrolidin-1-yl-carbonyl)-quinazoline
(179) 6-chloro-4-[1-(5-chloro-1H-benzimidazol-2-yl)-3-(N-methylaminocarbonyl-methyl-N-methyl-amino-carbonyl)-propyl-amino]-7-(pyrrolidin-1-yl-carbonyl)-quinazoline
(180) 6-chloro-4-{1-(5-chloro-1H-benzimidazol-2-yl)-3-[N-(2-(1H)-imidazol-4-yl)-ethyl)-N-methyl-amino-carbonyl]-propyl-amino}-7-(pyrrolidin-1-yl-carbonyl)-quinazoline
(181) 6-chloro-4-[1-(5-chloro-1H-benzimidazol-2-yl)-3-(1-thiazolidin-3-yl-carbonyl)-propyl-amino]-7-(pyrrolidin-1-yl-carbonyl)-quinazoline
(182) 6-chloro-4-[1-(5-chloro-1H-benzimidazol-2-yl)-3-(N-cyclopropyl-N-methyl-amino-carbonyl)-propyl-amino]-7-(pyrrolidin-1-yl-carbonyl)-quinazoline
(183) 6-chloro-4-[1-(5-chloro-1H-benzimidazol-2-yl)-3-(N-cyclopropylmethyl-N-methyl-amino-carbonyl)-propyl-amino]-7-(pyrrolidin-1-yl-carbonyl)-quinazoline
(184) 6-chloro-4-[1-(5-chloro-1H-benzimidazol-2-yl)-3-(cyclopentylamino-carbonyl)-propyl-amino]-7-(pyrrolidin-1-yl-carbonyl)-quinazoline
(185) 6-chloro-4-[1-(5-chloro-1H-benzimidazol-2-yl)-3-(N-piperidin-4-yl-amino-carbonyl)-propyl-amino]-7-(pyrrolidin-1-yl-carbonyl)-quinazoline
(186) 6-chloro-4-{1-(5-chloro-1H-benzimidazol-2-yl)-3-[C-(pyridin-2-yl)-methylamino-carbonyl]-propyl-amino}-7-(pyrrolidin-1-yl-carbonyl)-quinazoline
(187) 6-chloro-4-[(1S)-1-(5-chloro-1H-benzimidazol-2-yl)-3-hydroxycarbonyl-propylamino]-7-(pyrrolidin-1-yl-carbonyl)-quinazoline
(188) 6-chloro-4-[(1S)-1-(5-chloro-1H-benzimidazol-2-yl)-ethylamino]-7-(5,6,7,8-tetrahydro-[1,2,4]triazolo[4,3a]pyridin-4-yl)-quinazoline
(189) 6-chloro-4-[(1S)-1-(5-chloro-1H-benzimidazol-2-yl)-3-(1,1-dioxo-isothiazolidin-2-yl)-propyl-amino]-7-(2,5-dihydropyrrol-1-yl-carbonyl)-quinazoline
(190) 6-chloro-4-[(1S)-1-(5-chloro-1H-benzimidazol-2-yl)-3-methanesulphonylamino-propyl-amino]-7-(2,5-dihydropyrrol-1-yl-carbonyl)-quinazoline
(191) 4-[(1S)-1-(5-chloro-1H-benzimidazol-2-yl)-3-(methylsulphanyl)-propylamino]-6-methoxy-7-(2,5-dihydropyrrol-1-yl-carbonyl)-quinazoline
(192) 4-[(1S)-1-(5-chloro-1H-benzimidazol-2-yl)-2-methoxy-ethylamino]-6-methoxy-7-(2,5-dihydropyrrol-1-yl-carbonyl)-quinazoline
(193) 6-chloro-4-[(1S)-1-(5-chloro-1H-benzimidazol-2-yl)-2-methoxy-ethylamino]-7-(thiazolidinyl-carbonyl)-quinazoline
(194) 4-[(1S)-1-(5-chloro-1H-benzimidazol-2-yl)-2-methoxy-ethylamino]-6-methyl-7-(2,5-dihydropyrrol-1-yl-carbonyl)-quinazoline
(195) 4-[(1S)-1-(5-chloro-1H-benzimidazol-2-yl)-2-methoxy-ethylamino]-6-methyl-7-(thiazolidinyl-carbonyl)-quinazoline
(196) 6-bromo-4-[(1S)-1-(5-chloro-1H-benzimidazol-2-yl)-2-methoxy-ethylamino]-7-(2,5-dihydropyrrol-1-yl-carbonyl)-quinazoline
(197) 6-bromo-4-[(1S)-1-(5-chloro-1H-benzimidazol-2-yl)-2-methoxy-ethylamino]-7-(thiazolidinyl-carbonyl)-quinazoline
(198) 6-chloro-4-[(1S)-1-(5-chloro-1H-benzimidazol-2-yl)-ethylamino]-7-(6,7,8,9-tetrahydro-[1,2,4]triazolo[4,3-a]pyridin-4-yl)-quinazoline
(199) 6-chloro-4-{1-(5-chloro-1H-benzimidazol-2-yl)-3-[2-(pyridin-4-yl-amino)-ethylamino-carbonyl]-propylamino}-7-(pyrrolidin-1-yl-carbonyl)-quinazoline
(200) 4-[(1S)-1-(5-bromo-1H-benzimidazol-2-yl)-2-methoxy-ethylamino]-6-chloro-7-(2,5-dihydropyrrolyl-carbonyl)-quinazoline and
(201) 4-[(1S)-1-(5-bromo-1H-benzimidazol-2-yl)-ethylamino]-6-chloro-7-(2,5-dihydro-pyrrolyl-carbonyl)-quinazoline.
   or a pharmaceutically acceptable salt thereof.

Any reference to the above-mentioned compounds 2d within the scope of the present invention includes a reference to any pharmaceutically acceptable acid addition salts thereof which may exist. By the physiologically or pharmaceutically acceptable acid addition salts which may be formed from 2d are meant, according to the invention, pharmaceutically acceptable salts selected from among the salts of hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids.

Any reference to the abovementioned active ingredients 2d within the scope of the present invention includes a reference to any alkali metal and alkaline earth metal salts thereof which may exist. If the compounds 2d are present in the form of their basic salts, the sodium or potassium salts are particularly preferred.

The pharmaceutical combinations of 1 and 2d according to the invention are preferably administered by parenteral or oral route, the latter being particularly preferred. For oral or parenteral administration the pharmaceutical compositions according to the invention may be administered e.g. in the form of solutions and tablets.

### Pharmaceutical Compositions Comprising a Direct Thrombin Inhibitor 1 and an Fibrinogen Receptor Antagonists (Glycoprotein IIb/IIa Antagonists) 2e:

One embodiment of the invention is a pharmaceutical composition comprising an direct thrombin inhibitor 1 and an fibrinogen receptor antagonists (glycoprotein IIb/IIa antagonists) 2e. Binary compositions containing only one active 1 and one active 2e, optionally together with one or more pharmaceutically acceptable excipients or carriers, are preferred. In the pharmaceutical combinations according to the invention preferred fibrinogen receptor antagonists (glycoprotein IIb/IIa antagonists) 2e are selected from the group consisting of fradafiban, lefradafinban, abciximab (ReoPro), eptifibatide (Integrilin) and tirofiban (Aggrastat), optionally in the form of enantiomers, mixtures of enantiomers or the racemates.

Any reference to fibrinogen receptor antagonists (glycoprotein IIb/IIa antagonists) 2e within the scope of the present invention includes a reference to the salts, preferably pharmacologically acceptable acid addition salts, or derivatives which may be formed from the fibrinogen receptor antagonists. Examples of pharmacologically acceptable acid addition salts of the fibrinogen receptor antagonists (glycoprotein IIb/IIa antagonists) 2e according to the invention are the pharmaceutically acceptable salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. Preferred salts are selected from the group consisting of acetate, hydrochloride, hydrobromide, sulphate, phosphate, maleate and methanesulphonate.

Any reference to the abovementioned fibrinogen receptor antagonists (glycoprotein IIb/IIa antagonists) 2e within the scope of the present invention includes a reference to any alkali metal and alkaline earth metal salts thereof which may exist. If the compounds 2e are present in the form of their basic salts, the sodium or potassium salts are particularly preferred.

The pharmaceutical combinations of 1 and 2e according to the invention are preferably administered by parenteral or oral route, the latter being particularly preferred. For oral or parenteral administration the pharmaceutical compositions according to the invention may be administered e.g. in the form of solutions and tablets.

Suitable doses for the compounds 2e are:

abciximab: 0.25 mg/kg iv bolus+10 mcg/kg/h iv infusion
eptifibatide: 80-135 mcg/kg iv bolus+0.5-1.0 mcg/kg/min iv infusion
tirofiban: 0.15 mcg/kg/min

Suitable dosages for compounds 1 have already been given above.

### Pharmaceutical Compositions Comprising a Direct Thrombin Inhibitor 1 and an Vitamin K Antagonist 2f:

One embodiment of the invention is a pharmaceutical composition comprising an direct thrombin inhibitor 1 and Vitamin K antagonists 2f. Binary compositions containing only one active 1 and one active 2f, optionally together with one or more pharmaceutically acceptable excipients or carriers, are preferred. In the pharmaceutical combinations according to the invention preferred Vitamin K antagonists 2f are selected from the group consisting of Warfarin and Phenprocoumon, optionally in the form of enantiomers, mixtures of enantiomers or the racemates.

Any reference to Vitamin K antagonists 2f within the scope of the present invention includes a reference to the salts, preferably pharmacologically acceptable salts, or derivatives which may be formed from the Vitamin K antagonists. Examples of pharmacologically acceptable salts of the Vitamin K antagonists 2f according to the invention is the sodium salt.

Any reference to the abovementioned Vitamin K antagonists 2f within the scope of the present invention includes a reference to any alkali metal and alkaline earth metal salts thereof which may exist. If the compounds 2f are present in the form of their basic salts, the sodium or potassium salts are particularly preferred.

The pharmaceutical combinations of 1 and 2f according to the invention are preferably administered by parenteral or oral route, the latter being particularly preferred. For oral or parenteral administration the pharmaceutical compositions according to the invention may be administered e.g. in the form of solutions and tablets.

Suitable doses for the compounds 2f are:

Warfarin (sodium salt): 5 mg tablets
Phenprocoumon: 3 mg tablets

Suitable dosages for compounds 1 have already been given above.

The actives of the combinations according to the invention may be administered simultaneously, separately or sequentially. The preferred route of administration depends on the indication to be treated. Both components 1 and 2 may be administered orally, intravenously, subcutaneously, topically or rectally, using suitable formulations known in the art, such as tablets, coated tablets, pills, granules or granular powder, syrups, emulsions, suspensions, solutions, ointments, transdermal patches or suppositories, optionally together with inert and non-toxic pharmaceutically acceptable excipients or solvents.

The compositions according to the invention may be given for instance orally, intravenously, subcutaneously, by intramuscular injection, intraperitoneally, intranasally or transdermally, using suitable formulations known in the art, such as tablets, coated tablets, pills, capsules, granules or granular powder, aerosols, syrups, emulsions, suspensions, powders, solutions or transdermal patches, optionally together with inert and non-toxic pharmaceutically acceptable excipients or solvents.

Within the scope of the present invention, the term carrier may optionally be used instead of the term excipient.

The preparations according to the invention may contain the combination of active substances 1 and 2 either together in one formulation or in two separate formulations. These formulations which may be used within the scope of the present invention are described in more detail in the next part of the specification.

Any aforementioned possible doses applicable for the combinations according to the invention are to be understood as referring to doses per single application. However, these examples are not be understood as excluding the possibility of administering the combinations according to the invention multiple times. Depending on the medical need patients may receive also multiple applications. As an example patients may receive the combinations according to the invention for instance two or three times in the morning of each treatment day. As the aforementioned dose examples are only to be understood as dose examples per single application multiple application of the combinations according to the invention leads to multiple doses of the aforementioned examples. The application of the compositions according to the invention can be for instance once a day, or depending on the duration of action of the agents twice a day, or once every 2 or 3 days.

The Examples which follow serve to illustrate the present invention in more detail without restricting the scope of the invention to the following embodiments by way of example.

### EXAMPLES OF FORMULATIONS

The following examples of formulations, which may be obtained analogously to methods known in the art, serve to illustrate the present invention more fully without restricting it to the contents of these examples. Examples of formulations comprising an direct thrombin inhibitor 1 selected from compounds 1.1 to 1.8 as the only active ingredient are disclosed in the prior art, e.g. in WO 98/37075 and WO 04/014894.

Additionally, suitable formulations for a drug may be the formulations disclosed in Rote Liste® 2005, Editio Cantor Verlag Aulendorf, Germany, or in Physician's Desk Reference, 58 edition, 2004.

### Example 1

### Dry Ampoule Containing 75 mg of Active Substance Per 10 ml

### Composition:

| | | |
|---|---|---|
| Active substance | 75.0 | mg |
| Mannitol | 50.0 | mg |
| water for injections | ad 10.0 | ml |

### Preparation:

Active substance and mannitol are dissolved in water. After packaging the solution is freeze-dried. To produce the solution ready for use, the product is dissolved in water for injections.

### Example 2

### Dry Ampoule Containing 35 mg of Active Substance Per 2 ml

### Composition:

| | | |
|---|---|---|
| Active substance | 35.0 | mg |
| Mannitol | 100.0 | mg |
| water for injections | ad 2.0 | ml |

### Preparation:

Active substance and mannitol are dissolved in water. After packaging, the solution is freeze-dried.

To produce the solution ready for use, the product is dissolved in water for injections.

### Example 3

### Tablet Containing 50 mg of Active Substance

### Composition:

| | |
|---|---|
| (1) Active substance | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

### Preparation:

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 9 mm.

### Example 4

### Tablet Containing 350 mg of Active Substance

### Preparation:

| | |
|---|---|
| (1) Active substance | 350.0 mg |
| (2) Lactose | 136.0 mg |
| (3) Maize starch | 80.0 mg |
| (4) Polyvinylpyrrolidone | 30.0 mg |
| (5) Magnesium stearate | 4.0 mg |
| | 600.0 mg |

(1), (2) and (3) are mixed together and granulated with an aqueous solution of (4). (5) is added to the dried granulated material. From this mixture tablets are pressed, biplanar, faceted on both sides and with a dividing notch on one side.

Diameter of the tablets: 12 mm.

### Example 5

### Capsules Containing 50 mg of Active Substance

### Composition:

| | |
|---|---|
| (1) Active substance | 50.0 mg |
| (2) Dried maize starch | 58.0 mg |
| (3) Powdered lactose | 50.0 mg |
| (4) Magnesium stearate | 2.0 mg |
| | 160.0 mg |

### Preparation:

(1) is triturated with (3). This trituration is added to the mixture of (2) and (4) with vigorous mixing.

This powder mixture is packed into size 3 hard gelatin capsules in a capsule filling machine.

### Example 6

### Capsules Containing 350 mg of Active Substance

### Composition:

| | |
|---|---|
| (1) Active substance | 350.0 mg |
| (2) Dried maize starch | 46.0 mg |
| (3) Powdered lactose | 30.0 mg |
| (4) Magnesium stearate | 4.0 mg |
| | 430.0 mg |

### Preparation:

(1) is triturated with (3). This trituration is added to the mixture of (2) and (4) with vigorous mixing.

This powder mixture is packed into size 0 hard gelatin capsules in a capsule filling machine.

### Example 7

### Suppositories Containing 100 mg of Active Substance

1 suppository contains:

| | |
|---|---|
| Active substance | 100.0 mg |
| Polyethyleneglycol (M.W. 1500) | 600.0 mg |
| Polyethyleneglycol (M.W. 6000) | 460.0 mg |
| Polyethylenesorbitan monostearate | 840.0 mg |
| | 2,000.0 mg |

Example 8 and 9 are formulation particularly adapted for the methanesulfonate of compound 1.1. A detailed description of the preparation thereof is given in WO 03/074056, which is hereby incorporated by reference.

### Example 8

### Pellets for Capsules

| | percentage composition | | | | | |
|---|---|---|---|---|---|---|
| | | | active | | per | per |
| | | insulating | substance | | capsule | capsule |
| | core material | layer | layer | total | [mg] | [mg] |
| tartaric acid | 61.3 | — | — | 61.3 | 176.7 | 353.4 |
| gum arabic | 3.1 | 2.8 | | 5.9 | 17.0 | 34.0 |
| talc | — | 5.6 | 3.2 | 8.8 | 25.4 | 50.7 |
| hydroxypropylcellulose | — | — | 4.0 | 4.0 | 11.5 | 23.1 |
| active substance | — | — | 20.0 | 20.0 | 57.7* | 115.3** |
| total | | | | 100.0 | 288.3 | 576.5 |

*corresponds to 50 mg of the compound of the active substance base
**corresponds to 100 mg of the compound of the active substance base

### Example 9

### Pellets for Capsules

| | percentage composition | | | | | |
|---|---|---|---|---|---|---|
| | | | active | | per | per |
| | | insulating | substance | | capsule | capsule |
| | core material | layer | layer | total | [mg] | [mg] |
| tartaric acid | 38.5 | — | — | 38.5 | 55.5 | 166.5 |
| gum arabic | 1.9 | 1.7 | | 3.6 | 5.2 | 15.6 |
| talc | — | 3.5 | 6.4 | 9.9 | 14.3 | 42.8 |
| hydroxypropylcellulose | — | — | 8.0 | 8.0 | 11.5 | 34.6 |
| active substance | — | — | 40.0 | 40.0 | 57.7* | 173.0** |
| total | | | | 100.0 | 144.2 | 432.5 |

*corresponds to 50 mg of the compound of the active substance base
**corresponds to 150 mg of the compound of the active substance base

1. A pharmaceutical composition comprising a direct thrombin inhibitor 1.1
(1.1) ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (dabigatran),
optionally in the form of tautomers, racemates, enantiomers, diastereomers, pharmacologically acceptable acid addition salts, or prodrugs thereof,
and one or more platelet inhibitors 2a, wherein the platelet inhibitor 2a is selected from the group consisting of acetylsalicylic acid 2a.1, clopidogrel 2a.2 and ticlopidine 2a.3, optionally in the form of the racemates, the enantiomers, the diastereomers and optionally the pharmacologically acceptable acid addition salts and the hydrates thereof and
optionally together with one or more pharmaceutically acceptable excipients or carriers.

2-8. (canceled)

9. The pharmaceutical composition of claim 1, wherein said composition is a ternary combination comprising the direct thrombin inhibitor 1.1 and two active compounds selected from the group consisting of acetylsalicylic acid 2a.1, clopidogrel 2a.2 and ticlopidine 2a.3 optionally together with one or more pharmaceutically acceptable excipients or carriers.

10-14. (canceled)

15. The pharmaceutical composition of claim 1, wherein the direct thrombin inhibitor is the methanesulfonate of compound 1.1.

16. The pharmaceutical composition of claim 1 or 15, wherein said composition is a binary composition and the platelet inhibitor is acetylsalicylic acid 2a.1.

17. The pharmaceutical composition of claim 1 or 15, wherein the platelet inhibitor is clopidogrel 2a.2.

18. (canceled)

19. The pharmaceutical composition according to claim 1 or 15, wherein said composition is a binary composition comprising a methanesulfonate of compound 1.1 and clopidogrel 2a.2.

20. The ternary pharmaceutical composition according to claim 9, wherein the composition comprises a methanesulfonate of compound 1.1 acetylsalicylic acid 2a.1 and clopidogrel 2a.2.

21. The pharmaceutical composition according to any one of claims 1, 15 and 16, wherein said composition is in a dosage form suitable for inhalative, oral, intravenous, topical, subcutaneous, intramuscular, intraperitoneal, intranasal, transdermal or rectal administration.

22. The pharmaceutical composition of claim 21, wherein said dosage from is suitable for oral administration.

23. The pharmaceutical composition according to claim 21, wherein said dosage form is suitable for intravenous administration.

24. The pharmaceutical composition according to claim 21, wherein said dosage form is suitable for subcutaneous administration.

25. A method for preventing or treating the consequences of thrombotic and thromboembolic diseases comprising administering to a patient in need thereof a therapeutically effective amount of pharmaceutical composition according to any of claims 1, 9, 15-17 and 19-24.

26. The method according to claim 25 wherein the thrombotic or thromboembolic disease is selected from the following indications:
(a) deep vein thrombosis (DVT) pulmonary embolism, and other venous thrombotic events in patients at risk for such events (post-orthopedic surgery, medical patients, cancer patients, surgical patients);
(b) stroke prevention in atrial fibrillation (SPAF);
(c) stroke prevention in other populations at high risk for such events (heart failure or left ventricular dysfunction, high risk patients with myocardial infarction, patients with valve disease or valve replacement);
(d) thrombosis and thombotic events in patients with acute myocardial infarction or acute coronary syndromes, including patients undergoing thrombolysis or those with stents or percutaneous coronary intervention (PCI), or both;
(e) post-myocardial infarction (MI), in patients who have received thrombolysis or those with percutaneous coronary intervention or post coronary bypass surgery;
(f) other acute coronary syndromes;
(g) thrombosis, preferably thrombosis in patients with stents or percutaneous coronary intervention (PCI).

27. The method of claim 26, wherein the indication is selected from DVT and SPAF.
